(19) United States
(12) Patent Application Publication
(10) Pub. No.: US 2025/0308646 A1
(43) Pub. Date: Oct. 2, 2025

(54) METHOD, DEVICE, TERMINAL EQUIPMENT AND STORAGE MEDIUM OF SLEEP EVALUATION

(71) Applicant: Guizhou Youpin sleep health industry Co., LTD, Tongren (CN)

(72) Inventors: Enping LIU, Tongren (CN); Yidi LIU, Tongren (CN); Ping TAN, Tongren (CN); Sujun LIU, Tongren (CN); Shengxiang WANG, Tongren (CN)

(73) Assignee: Guizhou Youpin sleep health industry Co., LTD, Tongren (CN)

(21) Appl. No.: 18/616,207

(22) Filed: Mar. 26, 2024

(51) Int. Cl. G16H 10/20; A61B 5/00; G16H 15/00

(52) U.S. Cl. CPC G16H 10/20; A61B 5/4806; G16H 15/00

(57) ABSTRACT

A method, a sleep evaluation device, a terminal device, and a storage medium of sleep evaluation include the following steps: responding to an evaluation list acquisition request sent by the client according to the main page of the sleep evaluation system, and returning the evaluation list to the client; responding to a test request sent by the client according to the evaluation items in the evaluation list, and returning a test question sheet corresponding to the evaluation items to the client; receiving an answer sheet uploaded by the client according to the test question sheet, quantifying the answer sheet, further inputting the quantized answer sheet into the trained NN neural network for score evaluation, and outputting an evaluation report to return the evaluation report to the client.

### TECHNICAL FIELD

The present disclosure relates to the field of sleep monitoring technology, in particular to a method, a device, a terminal equipment and a storage medium of sleep evaluation.

### BACKGROUND

A reasonable lifestyle is a key factor in ensuring human health and an effective way to prevent various physiological and psychological diseases. Lifestyle is generally reflected in a person's diet, exercise, and sleep. At present, there are many intelligent evaluation methods for diet and exercise, such as diet evaluation through dietary survey and evaluation system, and exercise evaluation through smart bracelets, but there is no effective means to evaluate sleep quality.

In order to solve the problem of sleep quality evaluation, in the prior art, usually, the body movement of the human body in the sleep state monitored by the acceleration sensor is used as the sleep monitoring parameter for sleep evaluation, or pressure sensor is used to monitor the heart rate and respiratory rate for sleep evaluation. The above sleep assessment methods rely on simple logical processing by sensors and processors, but in fact, due to the complexity and particularity of sleep habits and human constitution, it is difficult for users to accurately perceive whether the above assessment methods really meet their own purpose of assessing sleep quality only by relying on physical experience

### SUMMARY

The embodiment of the present disclosure is to provide a method of sleep evaluation that combines self perception evaluation with artificial intelligence to improve the accuracy of sleep quality evaluation.

A method of sleep evaluation provided by the embodiment of the present disclosure includes:
responding to an evaluation list acquisition request sent by a client according to a main page of a sleep evaluation system, and returning the evaluation list to the client;
responding to a test request sent by the client according to evaluation items in the evaluation list, and returning a test question sheet corresponding to the evaluation items to the client; and
receiving an answer sheet uploaded by the client according to the test question sheet, quantifying the answer sheet, further inputting the quantized answer sheet into a trained NN neural network for score evaluation, and outputting an evaluation report to return the evaluation report to the client.

Further, the step of quantifying the answer sheet specifically includes:
carrying out quantization processing on each question in that answer sheet by adopt a matrix sparse coding mode to obtain a coding matrix corresponding to the answer sheet.

Further, the NN neural network includes an input layer, a hidden layer and an output layer; the input layer inputs an answer data of the quantized answer sheet;
the hidden layer adopts a three-layer structure and is internally provided with a plurality of neural units for autonomous learning and evaluating and analyzing for the answer data; and
the output layer outputs a score result of the answer sheet, that is, the evaluation report.

Further, the NN neural network adopts a deep learning neural network for model training, specifically, a multilayer NN network is configured to train the scoring mechanism of the test question sheet of each evaluation item in the evaluation list, and a training result is configured to fit an existing evaluation scoring formula, and further, the scoring mechanism is corrected according to a fitting result.

Further, the device of sleep assessment includes:
an Apache server, configured to process and verify the evaluation list acquisition request of the client and receive the answer sheet uploaded by the client;
a Node. JS server, configured to asynchronously process the answer data of the answer sheet and perform secondary verification on the answer data; and
a Python server, configured to execute an AI analysis of the answer sheet and return and provide answers to the Node. JS server.

The embodiment of the present disclosure has the following beneficial effects:
the embodiments of the present disclosure provide a method which includes the following steps: responding to an evaluation list acquisition request sent by the client and sending the evaluation list to the client; Receiving the answer sheet uploaded by the client, quantifying the answer sheet, inputting the trained NN neural network for score evaluation, and outputting an evaluation report. Compared to existing methods that rely on sensors and processors for simple processing to obtain sleep quality evaluation, the present disclosure combines self perception evaluation with artificial intelligence to improve the accuracy of sleep quality evaluation.

Further, another embodiment of the present disclosure provides a device of sleep assessment which includes:
an evaluation list sending module, configured to respond to an evaluation list acquisition request sent by a client according to a main page of a sleep evaluation system, and return the evaluation list to the client;
a test question sheet sending module, configured to respond to a test request sent by the client according to evaluation items in the evaluation list, and return a test question sheet corresponding to the evaluation items to the client; and
an evaluation report generation module, configured to receive an answer sheet uploaded by the client according to the test question sheet, quantify the answer sheet, further input the quantized answer sheet into a trained NN neural network for score evaluation, and output an evaluation report to return the evaluation report to the client.

Further, the step of quantifying the answer sheet specifically includes:
carrying out quantization processing on each question in that answer sheet by adopt a matrix sparse coding mode to obtain a coding matrix corresponding to the answer sheet.

Further, the NN neural network includes an input layer, a hidden layer and an output layer. the input layer inputs an answer data of the quantized answer sheet;
the hidden layer adopts a three-layer structure and is internally provided with a plurality of neural units for autonomous learning and evaluating and analyzing for the answer data; and
the output layer outputs a score result of the answer sheet, that is, the evaluation report.

Further, the NN neural network adopts a deep learning neural network for model training, specifically, a multilayer NN network is configured to train the scoring mechanism of the test question sheet of each evaluation item in the evaluation list, and a training result is configured to fit an existing evaluation scoring formula, and further, the scoring mechanism is corrected according to a fitting result.

Further, the embodiment of the present disclosure also provides a terminal equipment of sleep assessment, which includes a processor, a memory, and a computer program stored in the memory and configured to be executed by the processor; the processor implements the method of sleep assessment according to any one of claims 1 to 5 when executing the computer program.

The embodiment of the present disclosure has the following beneficial effects:
the embodiments of the present disclosure provide a method, a sleep evaluation device, a terminal device and a storage medium of sleep evaluation. The method includes the following steps: responding to an evaluation list acquisition request sent by the client and sending the evaluation list to the client; Receiving the answer sheet uploaded by the client, quantifying the answer sheet, inputting the trained NN neural network for score evaluation, and outputting an evaluation report. Compared to existing methods that rely on sensors and processors for simple processing to obtain sleep quality evaluation, the present disclosure combines self perception evaluation with artificial intelligence to improve the accuracy of sleep quality evaluation

### BRIEF DESCRIPTION OF THE DRAWINGS

FIG. 1 is a flowchart of an embodiment of the method of sleep evaluation provided by the present disclosure;

FIG. 2 is a flowchart of another embodiment of the method of sleep evaluation provided by the present disclosure;

FIG. 3 is a flowchart of another embodiment of the method of sleep evaluation provided by the present disclosure;

FIG. 4 is a structure diagram of an embodiment of the device of sleep evaluation provided by the present disclosure; and

FIG. 5 is a structure diagram of an embodiment of the multilayer NN network provided by the present disclosure.

### DETAILED DESCRIPTION OF THE EMBODIMENTS

In the following, the technical solutions of the embodiments of the present disclosure will be clearly and completely described in conjunction with the accompanying drawings of the embodiments of the present disclosure. Obviously, the described embodiments are only a part of embodiments, but not all embodiments. Based on the embodiments of the present disclosure, all other embodiments obtained by those of ordinary skill in the art without creative work are within the scope of the present disclosure.

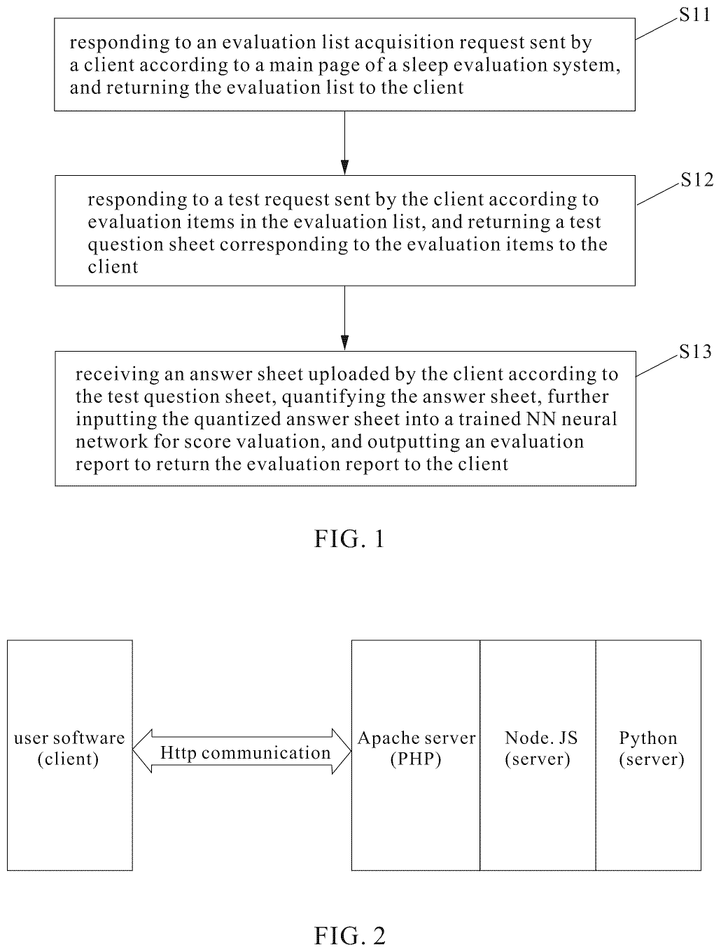

### Refer to FIGS. 1-3.

Referring to FIG. 1, it is a flowchart of an embodiment of the method of sleep evaluation provided by the present disclosure. As shown in FIG. 1, the diagnosis method includes steps S11 to S13. The steps are as follows:
Step S11: responding to an evaluation list acquisition request sent by a client according to a main page of a sleep evaluation system, and returning the evaluation list to the client;
Step S12: responding to a test request sent by the client according to evaluation items in the evaluation list, and returning a test question sheet corresponding to the evaluation items to the client; and
Step S13: receiving an answer sheet uploaded by the client according to the test question sheet, quantifying the answer sheet, further inputting the quantized answer sheet into a trained NN neural network for score evaluation, and outputting an evaluation report to return the evaluation report to the client.

In this embodiment, Step S11 is specifically to respond to an evaluation list acquisition request initiated by the client to the sleep evaluation system through Http communication, and return the evaluation list to the client, so that the client displays the inventory list to the user for answering.

In this embodiment, the client has two presentation modes, which are Windows software and browser software.

In this embodiment, the sleep evaluation system is a WEB server which includes: an Apache server, configured to process and verify the evaluation list acquisition request of the client and receive the answer sheet uploaded by the client, a Node. JS server, configured to asynchronously process the answer data of the answer sheet and perform secondary verification on the answer data; and a Python server, configured to execute an AI analysis of the answer sheet and return and provide answers to the Node. JS server.

In this embodiment, the evaluation list is divided into categories such as mental state evaluation, lifestyle evaluation, infant adjustment, and sleep duration. The client initiates a corresponding evaluation list acquisition request to the WEB server according to the evaluation lists in different evaluation categories selected by the user.

In this embodiment, the Step S12 includes responding, by the WEB server, the test request of the depression screening and evaluation list sent by the client, and returns the depression test list corresponding to the depression screening and evaluation list to the client for the user to perform self-evaluation.

In this embodiment, the Step S13 includes receiving, by the WEB server, a test question answer sheet for depression uploaded by the client according to the test question sheet for depression, quantizing each question in the test question answer sheet for depression by using a 9*4 matrix sparse coding manner, further inputting the quantized answer sheet into the trained NN neural network for recognition, and output the evaluation score, that is, the evaluation report to the client.

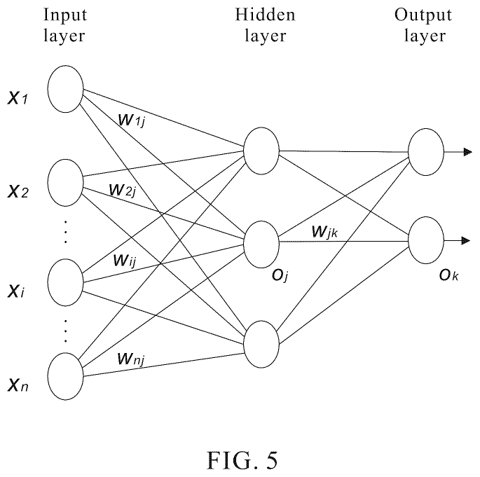

In this embodiment, the NN neural network includes an input layer, a hidden layer and an output layer. The input layer is configured to input an answer data of the quantized answer sheet as shown in FIG. 5 for x₁, x₂ . . . xₙ. The hidden layer adopts a three-layer structure and is internally provided with a plurality of neural units for autonomous learning and evaluating and analyzing for the answer data. The output layer is configured to output a score result of the answer sheet, that is, the evaluation report.

In this embodiment, the NN neural network adopts a deep learning neural network for model training. Specifically, the NN network is configured to train the scoring mechanism of the test question sheet of each evaluation item in the evaluation list, and a training result is configured to fit an existing evaluation scoring formula, and further, the scoring mechanism is corrected according to a fitting result until the evaluation score of the NN neural network is compared with the existing evaluation scoring formula and the error is less than 0.001%, and then the network training of the NN neural network is stopped.

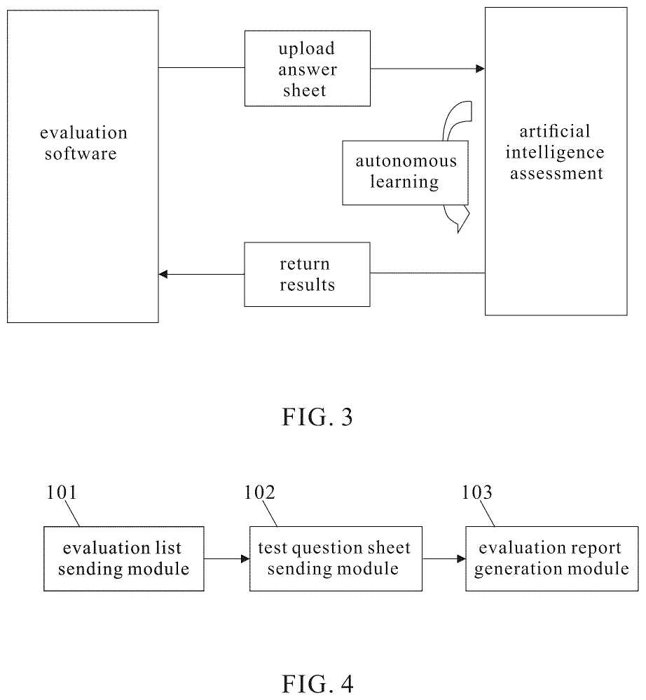

### Refer to FIG. 4.

Further, referring to FIG. 4, it is a structure diagram of an embodiment of the device of sleep evaluation provided by the present disclosure. As shown in FIG. 4, the device includes:

An evaluation list sending module 101, configured to respond to an evaluation list acquisition request sent by a client according to a main page of a sleep evaluation system, and return the evaluation list to the client.

In this embodiment, the evaluation list sending module 101 is configured to make the sleep evaluation system respond to an evaluation list acquisition request initiated by the client to the sleep evaluation system through Http communication, and return the evaluation list to the client, so that the client displays the inventory list to the user for answering.

In this embodiment, the client has two presentation modes, which are Windows software and browser software.

In this embodiment, the sleep evaluation system is a WEB server which includes: an Apache server, configured to process and verify the evaluation list acquisition request of the client and receive the answer sheet uploaded by the client, a Node. JS server, configured to asynchronously process the answer data of the answer sheet and perform secondary verification on the answer data; and a Python server, configured to execute an AI analysis of the answer sheet and return and provide answers to the Node. JS server.

In this embodiment, the evaluation list is divided into categories such as mental state evaluation, lifestyle evaluation, infant adjustment, and sleep duration. The client initiates a corresponding evaluation list acquisition request to the WEB server according to the evaluation lists in different evaluation categories selected by the user.

A test question sheet sending module 102, configured to respond to a test request sent by the client according to evaluation items in the evaluation list, and return a test question sheet corresponding to the evaluation items to the client.

In this embodiment, the test question sheet sending module 102 is configured to make the WEB server respond the test request of the depression screening and evaluation list sent by the client, and returns the depression test list corresponding to the depression screening and evaluation list to the client for the user to perform self-evaluation.

An evaluation report generation module 103, configured to receive an answer sheet uploaded by the client according to the test question sheet, quantify the answer sheet, further input the quantized answer sheet into a trained NN neural network for score evaluation, and output an evaluation report to return the evaluation report to the client.

In this embodiment, the evaluation report generation module 103 is configured to make the WEB server receive the test request of the depression screening and evaluation list sent by the client, and returns the depression test list corresponding to the depression screening and evaluation list to the client for the user to perform self-evaluation.

In this embodiment, the NN neural network includes an input layer, a hidden layer and an output layer. The input layer is configured to input an answer data of the quantized answer sheet as shown in FIG. 5 for x₁, x₂ . . . xₙ. The hidden layer adopts a three-layer structure and is internally provided with a plurality of neural units for autonomous learning and evaluating and analyzing for the answer data. The output layer is configured to output a score result of the answer sheet, that is, the evaluation report.

In this embodiment, the NN neural network adopts a deep learning neural network for model training. Specifically, the NN network is configured to train the scoring mechanism of the test question sheet of each evaluation item in the evaluation list, and a training result is configured to fit an existing evaluation scoring formula, and further, the scoring mechanism is corrected according to a fitting result until the evaluation score of the NN neural network is compared with the existing evaluation scoring formula and the error is less than 0.001%, and then the network training of the NN neural network is stopped.

It can be seen from the above that the embodiments of the present disclosure provide a method which includes the following steps: responding to an evaluation list acquisition request sent by the client and sending the evaluation list to the client; Receiving the answer sheet uploaded by the client, quantifying the answer sheet, inputting the trained NN neural network for score evaluation, and outputting an evaluation report. Compared to existing methods that rely on sensors and processors for simple processing to obtain sleep quality evaluation, the present disclosure combines self perception evaluation with artificial intelligence to improve the accuracy of sleep quality evaluation.

A person of ordinary skill in the art may understand that all or part of the processes for implementing the method of the above embodiments may be implemented by a computer program instructing related hardware, the program may be stored in a computer readable storage medium, and when executed, the program may include the processes of the above embodiments. The storage medium may be a magnetic disk, an optical disk, a read-only memory (Read-Only Memory, ROM), or a random access memory (Random Access Memory, RAM).

The above is the preferred embodiment of the present disclosure. It should be noted that, for those of ordinary skill in the art, several improvements and modifications can be made without departing from the principles of the present disclosure, and these improvements and modifications are also considered to be within the scope of protection of the disclosure.

What is claimed is:

1. A method of sleep assessment suitable for execution on computing equipment, comprising:
responding to an evaluation list acquisition request sent by a client according to a main page of a sleep evaluation system, and returning the evaluation list to the client;
responding to a test request sent by the client according to evaluation items in the evaluation list, and returning a test question sheet corresponding to the evaluation items to the client; and
receiving an answer sheet uploaded by the client according to the test question sheet, quantifying the answer sheet, further inputting the quantized answer sheet into a trained neural network for score evaluation, and outputting an evaluation report to return the evaluation report to the client.

2. The method of sleep assessment according to claim 1, wherein the step of quantifying the answer sheet further comprises:
carrying out quantization processing on each question in the answer sheet by adopt a matrix sparse coding mode to obtain a coding matrix corresponding to the answer sheet.

3. The method of sleep assessment according to claim 1, wherein the neural network comprises an input layer, a hidden layer and an output layer; wherein
the input layer inputs an answer data of the quantized answer sheet;
the hidden layer adopts a three-layer structure and is internally provided with a plurality of neural units for autonomous learning, evaluating, and analyzing for the answer data; and
the output layer outputs a score result of the answer sheet, that is, the evaluation report.

4. The method of sleep assessment according to claim 1, wherein the neural network adopts a deep learning neural network for model training, specifically, a multilayer neural network is configured to train the scoring mechanism of the test question sheet of each evaluation item in the evaluation list, and a training result is configured to fit an existing evaluation scoring formula, and further, the scoring mechanism is corrected according to a fitting result.

5. The method of sleep assessment according to claim 1, wherein the system of sleep assessment further comprises:
an Apache server, configured to process and verify the evaluation list acquisition request of the client and receive the answer sheet uploaded by the client;
a Node. JS server, configured to asynchronously process the answer data of the answer sheet and perform secondary verification on the answer data; and
a Python server, configured to execute an AI analysis of the answer sheet and return and provide answers to the Node. JS server.

6. A device of sleep assessment comprising:
an evaluation list sending module, configured to respond to an evaluation list acquisition request sent by a client according to a main page of a sleep evaluation system, and return the evaluation list to the client;
a test question sheet sending module, configured to respond to a test request sent by the client according to evaluation items in the evaluation list, and return a test question sheet corresponding to the evaluation items to the client; and
an evaluation report generation module, configured to receive an answer sheet uploaded by the client according to the test question sheet, quantify the answer sheet, further input the quantized answer sheet into a trained neural network for score evaluation, and output an evaluation report to return the evaluation report to the client.

7. The device of sleep assessment according to claim 6, wherein the step of quantifying the answer sheet further comprises:
carrying out quantization processing on each question in that answer sheet by adopting a matrix sparse coding mode to obtain a coding matrix corresponding to the answer sheet.

8. The device of sleep assessment according to claim 6, wherein the neural network comprises an input layer, a hidden layer and an output layer; wherein
the input layer inputs an answer data of the quantized answer sheet;
the hidden layer adopts a three-layer structure and is internally provided with a plurality of neural units for autonomous learning, evaluating, and analyzing for the answer data; and
the output layer outputs a score result of the answer sheet, that is, the evaluation report.

9. The device of sleep assessment according to claim 6, wherein the neural network adopts a deep learning neural network for model training, specifically, a multilayer neural network is configured to train the scoring mechanism of the test question sheet of each evaluation item in the evaluation list, and a training result is configured to fit an existing evaluation scoring formula, and further, the scoring mechanism is corrected according to a fitting result.

10. A terminal equipment of sleep assessment comprising: a processor, a memory, and a computer program stored in the memory and configured to be executed by the processor; the processor implements the method of sleep assessment according claim 1 when executing the computer program.

11. A terminal equipment of sleep assessment comprising: a processor, a memory, and a computer program stored in the memory and configured to be executed by the processor; the processor implements the method of sleep assessment according claim 2 when executing the computer program.

12. A terminal equipment of sleep assessment comprising: a processor, a memory, and a computer program stored in the memory and configured to be executed by the processor; the processor implements the method of sleep assessment according claim 3 when executing the computer program.

13. A terminal equipment of sleep assessment comprising: a processor, a memory, and a computer program stored in the memory and configured to be executed by the processor; the processor implements the method of sleep assessment according claim 4 when executing the computer program.

14. A terminal equipment of sleep assessment comprising: a processor, a memory, and a computer program stored in the memory and configured to be executed by the processor; the processor implements the method of sleep assessment according claim 5 when executing the computer program.
